(19) United States
(12) Patent Application Publication
(10) Pub. No.: US 2003/0077365 A1
(43) Pub. Date: Apr. 24, 2003

(54) Environmentally-friendly microbiological and microbiocidal control in aqueous systems

(76) Inventor: Jonathan N. Howarth, Baton Rouge, LA (US)

Correspondence Address: Mr. Philip M. Pippenger, Law Department, Albemarle Corporation, 451 Florida Street, Baton Rouge, LA, 70801-1765, US

(21) Appl. No.: 10/313,243

(22) Filed: Dec. 6, 2002

Related U.S. Application Data
(63) Continuation-in-part of application No. 10/029,329, filed on Dec. 21, 2001.
Application No. 10/029,329 is a continuation-in-part of application No. 09/893,581, filed on Jun. 28, 2001, now abandoned.

(51) Int. Cl. A23K 1/00

(52) U.S. Cl. 426/332

(57) ABSTRACT

In forming an aqueous microbiocidal solution, the solution is formed bypassing water through a bed of at least one 1,3-dibromo-5,5-dialkylhydantoin in non-powdery particulate form disposed in a feeder tank. Described are the particular 1,3-dibromo-5,5-dialkylhydantoins used and their compacted forms together with the particular design features of the feeder tank.

### REFERENCE TO RELATED APPLICATIONS

This is a continuation-in-part of commonly-owned copending application Ser. No. 10/029,329 filed Dec. 21, 2001, which is a continuation-in-part of application Ser. No. 09/893,581, filed Jun. 28, 2001, now abandoned.

### REFERENCE TO OTHER COMMONLY-OWNED APPLICATIONS

Reference is hereby made to the following commonly-owned applications: application Ser. No. 09/088,300, filed Jun. 1, 1998, now U.S. Pat. No. 6,068,861 issued May 30, 2000; application Ser. No. 09/296,499, filed Apr. 22, 1999, now U.S. Pat. No. 6,110,387 issued Aug. 29, 2000; application Ser. No. 09/323,348, filed Jun. 1, 1999, now U.S. Pat. No. 6,303,038 B1 issued Oct. 16, 2001; application Ser. No. 09/404,184, filed Sep. 24, 1999, now U.S. Pat. No. 6,322,822 B1 issued Nov. 27, 2001; application Ser. No. 09/442,025, filed Nov. 17, 1999, now U.S. Pat. No. 6,306,441 issued Oct. 23, 2001; application Ser. No. 09/451,319, filed Nov. 30, 1999; application Ser. No. 09/451,344, filed Nov. 30, 1999, now U.S. Pat. No. 6,352,725 B1 issued Mar. 5, 2002; application Ser. No. 09/456,781, filed Dec. 8, 1999; application Ser. No. 09/483,896, filed Jan. 18, 2000, now U.S. Pat. No. 6,448,410 B1 issued Sep. 10, 2002; application Ser. No. 09/484,687, filed Jan. 18, 2000; application Ser. No. 09/484,844, filed Jan. 18, 2000; application Ser. No. 09/484,891, filed Jan. 18, 2000; application Ser. No. 09/484,938, filed Jan. 18, 2000; application Ser. No. 09/487,816, filed Jan. 18, 2000; application Ser. No. 09/506,911, filed Feb. 18, 2000; application Ser. No. 09/658,839, filed Sep. 8, 2000, now U.S. Pat. No. 6,375,991 B1 issued Apr. 23, 2002; application Ser. No. 09/663,788, filed Sep. 18, 2000, now U.S. Pat. No. 6,348,291 B1 issued Feb. 19, 2002; application Ser. No. 09/663,948, filed Sep. 18, 2000, now U.S. Pat. No. 6,299,909 B1 issued Oct. 9, 2001; application Ser. No. 09/732,601, filed Dec. 7, 2000; application Ser. No. 09/775,516, filed Feb. 2, 2001; application Ser. No. 09/778,228, filed Feb. 6, 2001; application Ser. No. 09/785,890, filed Feb. 16, 2001; and application Ser. No. 09/974,622, filed Oct. 9, 2001.

### REFERENCE TO A JOINTLY-OWNED APPLICATION

Reference is hereby made to application Ser. No. 10/028,631, filed Dec. 21, 2001 of which one of two owners is the owner of the present application.

### TECHNICAL FIELD

This invention relates to new technology for effecting microbiological control in aqueous systems, such as industrial cooling water, recreational water, and water used in paper-manufacturing processes. More particularly, this invention relates to effectively and economically controlling both microorganisms and microbiocidal agent in such aqueous systems.

### BACKGROUND

Periodic slug dosing of recirculating water with high doses of biocide is a common way of maintaining microbiological control of the water. As an example, it is typical for a high dose of biocide to be applied to cooling tower water 3 to 4 times per week. In between dosing, the biocide is depleted in various ways, such as by reaction with, and eradication of, microorganisms, by biocide breakdown due to chemical instability, by evaporative flash-off, by physical removal in the form of blowdown and drift, or by chemical reaction of the biocide with contaminants or other components in the cooling water. As a result of such biocide depletion, microorganisms start to proliferate again. Therefore the ultimate goal of a slug dosing treatment program is to apply the next slug of biocide to eradicate these microorganisms before they build up to problematical levels.

Unfortunately, it is entirely possible that when using a slug dosing program to treat, say, cooling water, there will be periods when there is an insufficient microbiocidal level, or even no microbiocidal level, of the conventionally-used chlorine-based or bromine-based biocides. Disastrous consequences would ensue if an employee or member of the public contracted a disease such as Legionnaire's disease, from inhaling a spray of water, for example by merely walking past a cooling tower or decorative fountain infested with the pathogen. Thus the possibility of having periods where there is insufficient or even no microbiocide in the recirculating water is somewhat akin to playing Russian roulette.

To avoid this problem, use of continuous dosing of microbiocidal chemicals in water is becoming more common. In this way, the water is treated so as to always contain a suitably high concentration of the microbiocide. Organizations such as the Occupational Safety & Health Agency (OSHA) and the Cooling Technology Institute (CTI) have recommended a continuous feed of bromine or chlorine in quantity sufficient to maintain free chlorine residuals in the water of 0.5 to 1 ppm (as Cl₂) as the best practice for control of Legionella bacteria in the recirculating water.

Time-discharge release of biocides to water systems has been carried out by using either floating devices or chemical feeders wherein water is caused to flow through a containment of chemical to be dissolved. In use of such chemical feeders, a known type of device has a container through which water is pumped by, for example, a swimming pool filtration and circulation system for purposes of dissolving treatment chemical in the container.

Chlorination chemicals or other chemical compositions for treatment of swimming pool water, such as calcium hypochlorite and various other chemicals are available in the form of a concentrated pellet or tablet wherein the active ingredients are held by a matrix of inert materials. The matrix breaks down when exposed to water, causing dissolution and entrainment of the substance held in a container.

Automatic chlorinators using a form of chlorine, such as trichloroisocyanuric acid, have been used which flow water over the chlorine product at a controlled rate while the pool pump is running. The dissolved chlorine is then mixed with the pool water. Adding chlorine in this manner has a number of disadvantages, in that the product form is relatively expensive, it is strongly on the acid side, and the rate of adding the chlorine is difficult to control. Although a granular form of chlorine has been utilized, the granular form of chlorine is unsatisfactory for automatic chlorinating systems. One such system is described in U.S. Pat. No. 3,626,972. In the arrangement shown in the patent, the granular material is stored in a supply bin from which it is released by gravity to introduce a measured volume of granules into a measured volume of water each time the pump cycles. One of the problems with granular dispensers of the type disclosed in the prior art is that the granular chlorine in the presence of moisture insufficient to dissolve the granules tends to swell and harden into a crusty form which does not then feed out of the storage container properly.

If the biocidal agent is fed through an erosion feeder on a slug-feed basis, saturated solutions will be generated in the feeder between slug-feeds. In the case of biocidal agents such as trichloroisocyanuric acid and bromochlorodialkylhydantoins, such solutions may be acidic. This can lead to the generation of potentially harmful chlorine or bromine vapors which may be released when an operator opens the feeder to refill the biocidal agent.

Therefore it would be beneficial if a system could be developed which provides continuous or substantially continuous biocidal treatment of an aqueous medium by use of a biocidal agent that goes not cake, swell or clump when exposed to atmospheric moisture. In addition, it would be of benefit if such a system could be found which does not lower excessively the pH of the treated water when slug-dosing techniques are employed which permit extended contact between a portion of the treated water and the biocide.

### SUMMARY OF THE INVENTION

The foregoing needs, among others, are deemed to be fulfilled in accordance with this invention wherein compacted forms of the highly effective, bromine-based microbiocide, 1,3-dibromo-5,5-dialkylhydantoin, are disposed in a microbiocide feeder system without clumping of the 1,3-dibromo-5,5-dialkylhydantoin so that a controlled amount of microbiocide is released into the aqueous media. Further advantages of this invention are provision for consistently controlled release of microbiocide as well as improved flowability of microbiocide in the body of the dispenser. Because the invention is also effective for slug-dosing as in, for example, an industrial water treatment situation, if there is prolonged contact between a portion of treated water with the 1,3-dibromo-5,5-dialkylhydantoin in the feeder due to the dosing technique employed, the 1,3-dibromo-5,5-dialkylhydantoin does not lower the pH of the water excessively. This feature ensures that an operator who opens the feeder to replenish the 1,3-dibromo-5,5-dialkylhydantoin does not get exposed to potentially harmful amounts of halogen vapors.

In one of its embodiments this invention provides a process for forming an aqueous microbiocidal solution. The method comprises passing water into a tank and upwardly through a bed of microbiocide in the tank so that the aqueous microbiocidal solution is formed. The microbiocide is at least one 1,3-dibromo-5,5-dialkylhydantoin in which one of the alkyl groups is a methyl group and the other alkyl group contains in the range of 1 to about 4 carbon atoms. The preferred 1,3-dibromo-5,5-dialkylhydantoin is 1,3-dibromo-5,5-dimethylhydantoin. The preferred forms of 1,3-dibromo-5,5-dialkylhydantoin are either nuggets or granules with granules being particularly preferred.

In a preferred embodiment of this invention, the tank comprises a pressure sealable first port at an upper portion of the tank to allow replenishment of the microbiocide in the bed, the bed being disposed in a lower portion of the tank. More particularly preferred is a process wherein the passage of the water through the tank is from a dispensing second port in an upward direction of flow through the lower portion of the tank and through at least a portion of the bed, and thereafter in a substantially horizontal direction out of the tank through a third port disposed between the lower and the upper portions of the tank.

One embodiment of this invention provides a system for treating an aqueous medium with a microbiocide. The system comprises:

(A) a vertically elongated tank comprising (i) an upper portion comprising a pressure sealable first port, (ii) a lower portion comprising a dispensing port, and (iii) a third port disposed between the upper and lower portions;

(B) a bed of microbiocide, the microbiocide being at least one 1,3-dibromo-5,5-dialkylhydantoin in which one of the alkyl groups is a methyl group and the other alkyl group contains in the range of 1 to about 4 carbon atoms; and

(C) water to be treated with the microbiocide;

wherein when the bed is disposed in the tank through the first port, water is dispensed into the tank through the second port to flow upwardly through at least a portion of the bed so as to form an aqueous solution comprising a microbiocidally effective amount of the microbiocide, which solution flows substantially horizontally out of the tank through the third port.

In another of its embodiments this invention provides a method for forming an aqueous microbiocidal solution which method comprises:

(A) disposing a bed of a microbiocide, the microbiocide being at least one 1,3-dibromo-5,5-dialkylhydantoin in which one of the alkyl groups is a methyl group and the other alkyl group contains in the range of 1 to about 4 carbon atoms, into a lower portion of a vertically elongated tank though a pressure sealable first port at an upper portion of the tank;

(B) dispensing water through a second port in the lower portion of the tank;

(C) passing the water upwardly through at least a portion of the bed so as to form an aqueous microbiocidal solution; and

(D) diverting the aqueous microbiocidal solution from its upward flow so that it flows substantially horizontally out of the tank through a third port of the tank;

wherein the solution which exits the third port of the tank comprises a microbiocidally effective amount of the microbiocide.

### BRIEF DESCRIPTION OF THE DRAWING

FIG. 1 is a schematic view of an embodiment of the invention.

### FURTHER DETAILED DESCRIPTION OF THE INVENTION

Pursuant to this invention there is provided a way of forming aqueous microbiocidal solutions of one or more 1,3-dibromo-5,5-dialkylhydantoins in which one of the alkyl groups is a methyl group and the other alkyl group contains in the range of 1 to about 4 carbon atoms, most preferably 1,3-dibromo-5,5-dimethylhydantoin, (“dibromodialkylhydantoin(s)”) comprises passing water through a bed of one or more such dibromodialkylhydantoin(s) in granular, nugget, pellet, tablet or other non-powdery particulate form (“bed”) disposed in a canister, tank, or other similar vessel (“tank”). Preferably the tank has a pressure sealable port at its upper portion for periodically replenishing the contents of the bed, and the water is caused to flow upwardly through a portion of the bed. More preferably, the tank is elongated in an upward direction so that the bed is longer from top to bottom than from side to side, this upward water flow is dispensed into the bed to flow upwardly through only a lower portion of the bed, and thence substantially horizontally through a port disposed between the lower and the upper portions of the bed and tank. In this way the upper portion of the bed serves as a reserve supply of contents of the bed which automatically feeds into the lower portion of the bed under gravity as the lower portion of the bed is slowly but substantially uniformly dissolved away in the water flow. Thus in this operation the water flow is preferably at least a substantially continuous flow, and most preferably, is a continuous flow. Methods for producing granules, tablets or other non-powdery particulate forms of 1,3-dibromo-5,5-dimethylhydantoin are described in detail in commonly-owned copending applications PCT/US 01/01541, published on Jul. 26, 2001 as WO 0153215; PCT/US 01/01545, published on Jul. 26, 2001 as WO 0152651; and PCT/US 01/01585, published on Jul. 26, 2001 as WO 0152656; all filed Jan. 17, 2001, each claiming priority based on respective earlier-filed corresponding U.S. applications. Each of these documents referred to herein are incorporated herein by reference in toto as if fully set forth in this document. Excellent process technology for producing 1,3-dibromo-5,5-dimethylhydantoin for use in making such granules, tablets or other non-powdery particulate forms is described in detail in commonly-owned copending application PCT/US 01/01544, filed Jan. 17, 2001, now published on Jul. 26, 2001 as WO 0153270, claiming priority based on an earlier-filed corresponding U.S. application. The disclosures of each such PCT and U.S. application as referred to herein is incorporated herein by reference in toto as if fully set forth in this document. Particularly preferred apparatus for use in conjunction with such granules, tablets or other non-powdery particulate forms of these dibromodialkylhydantoin(s) in forming aqueous microbiocidal solutions thereof is available from Neptune Chemical Pump Company, a division of R. A. Industries, Inc., Lansdale, Pa. 19446, as “Bromine Feeders” Models BT-15, BT-40, BT-42, BT-80, BT-160, BT-270, and BT-350, or equivalent. Excellent results are achieved using combinations of Model BT-40 with granules of 1,3-dibromo-5,5-dimethylhydantoin (formally marketed by Albemarle Corporation under the trade designation of Albrom 100 biocide and now called Albrom 100PC disinfectant). Single charges of such microbiocides in tablet or granular form in such a device can provide continuous highly-effective microbiocidal activity in bodies of end use water at ordinary outdoor temperatures for as long as five (5) months without need for replenishment.

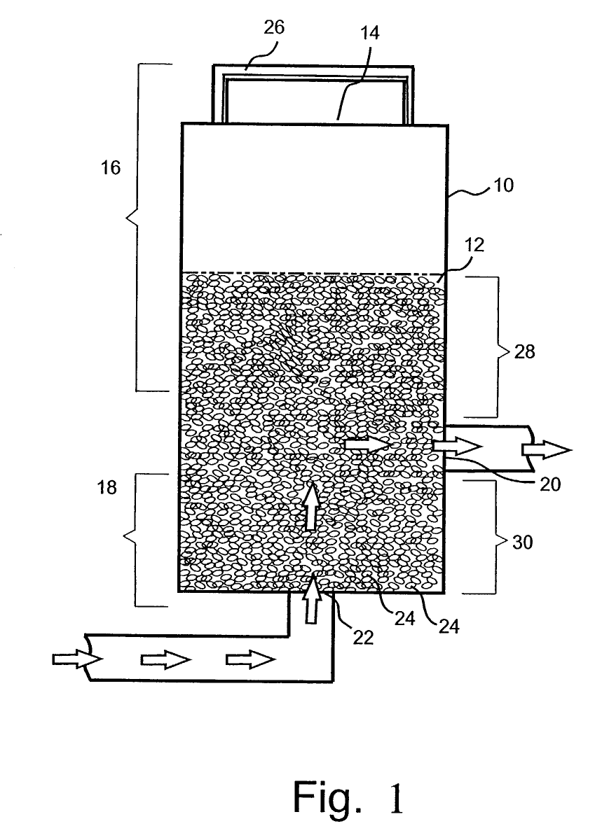

An embodiment of this invention is illustrated in FIG. 1 wherein an especially cost-effective, operationally efficient, and highly preferred way of forming aqueous microbiocidal solutions of one or more 1,3-dibromo-5,5-dialkylhydantoins is produced by passing water through a bed 12 of one or more such dibromodialkylhydantoin(s) in granular or nugget form. Bed 12, comprised of granules 24,24 is disposed in a tank 10 which has an elongated cylindrical shape which is longer from top to bottom than from side to side. Tank 10 has a pressure sealable first port 14 at an upper portion 16 of tank 10 for periodically replenishing the contents of the bed 12. Water is caused to flow upwardly through a lower portion 30 of bed 12, as shown generally by the direction of the arrows. This water flow is dispensed into tank 10 through a dispensing second port 22, into bed 12 and caused to flow upwardly through only lower portion 30 of bed 12. Thence water flows substantially horizontally through a third port 20 out of tank 10. Third port 20 is disposed between lower portion 18 and upper portion 16 of tank 10 and also respectively between lower portion 30 and upper portion 28 of bed 12. In this way upper portion 28 of bed 12 serves as a reserve supply of contents of bed 12 which automatically feeds into lower portion 30 of bed 12 under gravity as lower portion 30 of bed 12 is slowly but substantially uniformly dissolved away in the water flow. Water flow may be intermittent but is preferably at least a substantially continuous flow, and most preferably, is a continuous flow.

Pressure sealable first port 14 of tank 10 can be seen in FIG. 1 to be sealed by cover 26. Cover 26 is any suitable size and configuration so that access is provided to replenish bed 12 as needed. In addition, the form of closure of cover 26 is such that pressure created within tank 10 due to the normal passage of water there through does not cause leakage. Typically such closure can be accomplished, for example, by use of gaskets and snap-closures or by a threaded cover closing a threaded port.

Thus applying the microbiocidally-effective amounts of solid-state microbiocides of these embodiments of the invention causes the microbiocide to be leached into water streams passing through conduits and into the tank or other feeder devices utilized in the treatment of industrial and recreational water media. For example, suitable solid forms of the microbiocide, preferably a bromine-based microbiocide, such as tablets, pellets, nuggets, or granules are placed in suitable feeding devices through which a stream of water is passed. The passage of the water through the bed of the microbiocide results in the stream continuously dissolving small quantities of the microbiocide to thereby provide microbiocidally effective amounts of the microbiocide in the water. 1,3-Dibromo-5,5-dimethylhydantoin is especially preferred for use in this mode of application because of its relatively low solubility and thus relatively slow rate of dissolution in water at ambient room temperatures. This translates into relatively long periods of use before need of refilling the device holding the solids. By way of example, the solubility of 1,3-dibromo-5,5-dimethylhydantoin in water at 75° F. (ca. 24° C.) is 405 ppm expressed as Cl₂ whereas the solubilities of N,N′-bromochloro-5,5-dimethylhydantoin and of the commercial mixture of N,N′-bromochloro-5,5-dimethylhydantoin and 1,3-dichloro-5-ethyl-5-methylhydantoin at the same temperature are, respectively, 890 ppm and 1905 ppm, both expressed as Cl₂.

Such bromine-based microbiocides are more effective than chlorine-based microbiocides against various bacteria and biofilms. In addition, these bromine-based microbiocides tend to be less odorous than chlorine-based microbiocides. Moreover, while some of the bromine-based microbiocides may possibly react with nitrogenous species, such as are present in water, the resultant bromamines would also possess microbiological activity. Thus such side reactions would not materially decrease the microbiological effectiveness made available by use of these bromine-based microbiocides. Furthermore, bromamines generally do not exhibit obnoxious properties toward workers whereas chloramines resulting from use of certain chlorine-based microbiocides under the same conditions tend to be powerful lachrymators.

Microbiocidal agents used pursuant to this invention can be produced economically in straightforward processing from relatively low cost raw materials and because of their effectiveness, can provide microbiological control on an economical basis consistent with the needs of the industry.

As previously noted, apparatus for use in conjunction with the non-powdery particulate forms of these dibromodialkylhydantoin(s) in forming aqueous microbiocidal solutions thereof are available from Neptune Chemical Pump Company, Lansdale, Pa. Additional commercially available feeders which can be used as a component of the present invention include Hayward 100 and 200 series chlorinators and brominators, available from Hayward Pool Products, Inc., Elizabeth, N.J., Rainbow 320 chlorinators available from Pentair Pool Products, Inc., Moorpark, Calif., and BioLab brominators available from Houghton Chemical Corporation, Alston, Mass.

Motive force of the water flow of the aqueous medium through the embodiment of this invention can be provided by any of the typical pumping devices available in industrial water treatment and/or recreational water treatment systems.

In an embodiment of this invention the 1,3-dibromo-5,5-dialkylhydantoins solids are in the form of shapes comprised of agglomerated or compressed particles. Examples of such shapes are nuggets, granules, tablets, and the like. While there are no hard and fast rules governing differentiation with respect to size among nuggets, granules, and tablets, typically nuggets and granules are regarded as being particles typically ranging in size from about 80 to about 3 U.S. standard mesh size. Tablets typically fall in the range of from about 0.5 to about 1.0 inch in diameter and about 0.5 to about 1.0 inch in thickness. It will be understood and appreciated however, that the foregoing dimensions are illustrative and are not intended to unduly limit the scope of this invention.

Typically the particulate forms used in the bed initially have a particle size no smaller in any dimension than in the range of about 6 U.S. standard mesh and no larger than about 1 inch and less than about 10% by weight of the initial material having dimensions outside these ranges. The non-powdery particulate form of microbiocide can also be provided in the form of tablets having an average diameter in the range of about 0.5 inch to about 1 inch in diameter. Of the desirable forms of microbiocide of this invention, pellets having an average diameter in the range of about 0.1 inch to about 0.3 inch and an average length in the range of about 0.25 inch to about 0.75 inch are preferred. More preferred forms include granules and nuggets of 1,3-dibromo-5,5-dimethylhydantoin which may be produced by pressure compacting the 1,3-dibromo-5,5-dimethylhydantoin using compression rolls and then breaking the compacted sheet formed into suitably sized granules or nuggets, which are classified by screening into the desired size range. Nuggets having an average size in the range of about 0.15 inch to about 0.5 inch are preferred, with nuggets in the range of about 0.13 inch to about 0.31 inch most preferred. Granules in the range of 6 U.S. Standard mesh size to about 0.5 inch are preferred with granules in the range of 6 U.S. Standard mesh size to about 0.312 in most particularly preferred. The non-powdery particulate forms of microbiocide of this invention have a large enough particle size so that they do not clump within the bed, but drop smoothly as the lower portion of the bed is slowly eroded away.

Such compacted shapes erode at slow, but essentially constant rates when maintained in a constant or substantially constant flow of water. Particularly preferred compacted granules of 1,3-dibromo-5,5-dimethylhydantoin can overcome problems associated with finely divided 1,3-dibromo-5,5-dimethylhydantoin powders. These problems can include particles sticking together which inhibit transfer of the granules into feed hoppers and generation of a large amount of irritating, corrosive dusts during transfer. The compacted shapes of the granules, for example those of the more preferred 1,3-dibromo-5,5-dimethylhydantoin, provide excellent flow ability with in the body of the tank and also greatly diminish the amount of irritating dust typically encountered when refilling the tank with 1,3-dibromo-5,5-dimethylhydantoin.

In a particularly preferred embodiment of the invention compacted granules of 1,3-dibromo-5,5-dimethylhydantoin can be formed without the need to resort to the use of binders; thus the granules are essentially homogeneous particles of active ingredient. This means that the granules are essentially pure granules of 1,3-dibromo-5,5-dimethylhydantoin, thus contributing to a more efficient method of forming a microbiocidally effective aqueous solution without addition of extraneous compounds.

In a preferred embodiment of the invention, the 1,3-dibromo-5,5-dimethylhydantoin is in nugget form which is marketed by Albemarle Corporation under the trade designation of Albrom™ 100 PC disinfectant. Typical properties of these nuggets include the following: (1) the assay of the 1,3-dibromo-5,5-dimethylhydantoin is at least about 98.0 wt %; (2) available bromine (Br₂) measured in wt % is at least about 109; (3) the bulk density, expressed in packed lbs/gal at ambient temperature is approximately 11.3; (4) the melting point/decomposition temperature is >374° F.; (5) the solubility in water at 68° F. is about 0.1 wt %; (6) the pH of a slurry in water of about 1 wt % is about 6.6; and (7) the nuggets' average size is in the range of about 0.13 to about 0.31 inch.

In a more preferred embodiment of the invention, the 1,3-dibromo-5,5-dimethylhydantoin is in granular form which is marketed by Albemarle Corporation under the trade designation of XtraBrom™ 111 biocide. Typical properties of these granules include the following: (1) the assay of the 1,3-dibromo-5,5-dimethylhydantoin is at least about 98.0 wt %; (2) available bromine (Br₂) measured in wt % is at least about 111; (3) the packed bulk density, expressed in g/cm³ at ambient temperature is approximately 1.1; (4) the melting point/decomposition temperature is >374° F.; (5) the solubility in water at 68° F. is about 0.1 wt %; (6) the pH of a slurry in water of about 1 wt % is about 6.6; and (7) the granules' average size is in the range of about 6 U.S. Standard mesh to about 0.312 inch.

A preferred system for use in the practice of these embodiments of this invention is a bromine-based microbiocidal solution of a 1,3-dibromo-5,5-dialkylhydantoin in which one of the alkyl groups is a methyl group and the other alkyl group contains in the range of 1 to about 4 carbon atoms. Thus these preferred biocides comprise 1,3-dibromo-5,5-dimethylhydantoin, 1,3-dibromo-5-ethyl-5-methylhydantoin, 1,3-dibromo-5-n-propyl-5-methylhydantoin, 1,3-dibromo-5-isopropyl-5-methylhydantoin, 1,3-dibromo-5-n-butyl-5-methylhydantoin, 1,3-dibromo-5-isobutyl-5-methylhydantoin, 1,3-dibromo-5-sec-butyl-5-methylhydantoin, 1,3-dibromo-5-tert-butyl-5-methylhydantoin, and mixtures of any two or more of them. Of the mixtures of the foregoing biocides that can be used pursuant to this invention, it is preferred to use 1,3-dibromo-5,5-dimethylhydantoin as one of the components, with a mixture of 1,3-dibromo-5,5-dimethylhydantoin and 1,3-dibromo-5-ethyl-5-methylhydantoin being particularly preferred. The most preferred member of this group of microbiocides is 1,3-dibromo-5,5-dimethylhydantoin. This compound is available in the marketplace in tablet, nugget or granular forms under the trade designations Albrom™ 100T, Albrom™ 100PC disinfectant and XtraBrom™ 111 and XtraBrom™ 111T biocide (Albemarle Corporation).

Methods for producing 1,3-dibromo-5,5-dialkylhydantoins are known and reported in the literature.

The highly desirable result of producing a microbiocidally effective solution of bromine-based biocide of the present invention is made possible by the distinctive interrelationships of the feeder's characteristic upward flow of water through a portion of the bed of granules of 1,3-dibromo-5,5-dimethylhydantoin disposed therein together with the highly desirable features of the granules of 1,3-dibromo-5,5-dimethylhydantoin, themselves. These notable features of the granules of 1,3-dibromo-5,5-dimethylhydantoininclude the low solubility which, when augmented by the distinctive compacted shape, coordinate to produce a slow rate of erosion of the granules of 1,3-dibromo-5,5-dimethylhydantoin within the bed—even while maintaining a safe level of free halogen residual in the treated aqueous media. The water flow pattern of the feeder (into the feeder in an upward direction and then substantially horizontally out of the feeder after passing through a portion of the bed of the granules of 1,3-dibromo-5,5-dimethylhydantoin) when coordinated with the specific compacted shape granules of 1,3-dibromo-5,5-dimethylhydantoin, together act cooperatively to provide a system in which the upper portion of the bed of granules of 1,3-dibromo-5,5-dimethylhydantoin serves as a reserve supply of biocide. The particle shape of the biocide is particularly advantageous in this respect, since the granules of 1,3-dibromo-5,5-dimethylhydantoin tend to fall smoothly downward without clumping or clogging as the 1,3-dibromo-5,5-dimethylhydantoin in the lower portion of the bed is slowly dissolved away. This interplay of the characteristics of the feeder plus the shape of the 1,3-dibromo-5,5-dimethylhydantoin, whether that shape is granular, nugget, pellet or tablet, and the low solubility of 1,3-dibromo-5,5-dimethylhydantoin translates into a decreased frequency for replenishing of biocide in the feeder while simultaneously providing a continuously biocidally effective flow of water out of the system.

In situations where slug-dosing of the aqueous media using such a feeder design is indicated, the low solubility of 1,3-dibromo-5,5-dimethylhydantoin, especially when compacted into granules, nuggets, pellets, or tablets, ensures that the biocide does not erode too fast between dosing cycles. This collaborative blend of the described feeder and microbiocide features overcomes the problem of having the biocide dissolve into a mushy mass in the bottom of the feeder with resultant plugging of intake and outlet feeder lines.

During slug-dosing operation water is caused to flow intermittently through the tank in time cycles having periods of active flow alternated with static periods, such that the solution has a pH in the range of about 5 to about 8 when measured proximate in time to the beginning of the period of active flow. By proximate in time, it is to be understood that the pH measurement is taken just before the water flow is commenced in the active flow period so that the solution is at its most concentrated with microbiocide.

The standard DPD test for determination of low levels of active chlorine or bromine is based on classical test procedures devised by Palin in 1974. See A. T. Palin, “Analytical Control of Water Disinfection With Special Reference to Differential DPD Methods For Chlorine, Chlorine Dioxide, Bromine, Iodine and Ozone”,J. Inst. Water Eng., 1974, 28, 139. While there are various modernized versions of the Palin procedures, the recommended version of the test is fully described in Hach Water Analysis Handbook, 3rd edition, copyright 1997. The procedure for “total chlorine” (i.e., active chlorine) is identified in that publication as Method 8167 appearing on page 379, Briefly, the “total chlorine” test involves introducing to the dilute water sample containing active chlorine or bromine, a powder comprising DPD indicator powder, (i.e., N,N′-diethyldiphenylenediamine), KI, and a buffer. The active chlorine or bromine species present react(s) with KI to yield iodine species which turn the DPD indicator to red/pink. The intensity of the coloration depends upon the concentration of “total chlorine” species (i.e., active chlorine”) present in the sample. This intensity is measured by a calorimeter calibrated to transform the intensity reading into a “total chlorine” value in terms of mg/L Cl₂. If the active halogen present is active bromine, the result in terms of mg/L Cl₂ is multiplied by 2.25 to express the result in terms of mg/L Br₂ of active bromine.

In greater detail, the DPD test procedure is as follows:

1. To determine the amount of species present in the water which respond to the “total chlorine” test, the water sample should be analyzed within a few minutes of being taken, and preferably immediately upon being taken.

2. Hach Method 8167 for testing the amount of species present in the water sample which respond to the “total chlorine” test involves use of the Hach Model DR 2010 calorimeter. The stored program number for chlorine determinations is recalled by keying in “80” on the keyboard, followed by setting the absorbance wavelength to 530 nm by rotating the dial on the side of the instrument. Two identical sample cells are filled to the 10 mL mark with the water under investigation. One of the cells is arbitrarily chosen to be the blank. To the second cell, the contents of a DPD Total Chlorine Powder Pillow are added. This is shaken for 10-20 seconds to mix, as the development of a pink-red color indicates the presence of species in the water which respond positively to the DPD “total chlorine” test reagent. On the keypad, the SHIFT TIMER keys are depressed to commence a three minute reaction time. After three minutes the instrument beeps to signal the reaction is complete. Using the 10 mL cell riser, the blank sample cell is admitted to the sample compartment of the Hach Model DR 2010, and the shield is closed to prevent stray light effects. Then the ZERO key is depressed. After a few seconds, the display registers 0.00 mg/L Cl₂. Then, the blank sample cell used to zero the instrument is removed from the cell compartment of the Hach Model DR 2010 and replaced with the test sample to which the DPD “total chlorine” test reagent was added. The light shield is then closed as was done for the blank, and the READ key is depressed. The result, in mg/L Cl₂ is shown on the display within a few seconds. This is the “total chlorine” level of the water sample under investigation.

### EXAMPLE 1

A field study was conducted in a four-cell tower that supplies cooling capacity for a manufacturing plant in order to compare 1,3-bromochloro-5,5-dimethylhydantoin and 1,3-dibromo-5,5-dimethylhydantoin in providing effective microbiocidal amounts in an economical manner. The tower had experienced chronic slime development in two of the cells. These two cells were part of a poorly-designed expansion and received low water flow across the distribution deck. It is believed that this caused slime to build up in both the basin and the fill areas. Microbiological fouling in the basin and fill areas required a shut down for manual cleaning on three occasions within one year. Table 1 presents features of the plant cooling system.

TABLE 1
Features of the Manufacturing Plant Cooling System

| | |
|---|---|
| Cooling Capacity | 4 × 500 ton units |
| Fill | Mix of medium and high efficiency film |
| Total system contained | 16,000 gallons |
| volume | |
| Recirculation rate | 4,100 gallons per minute (gpm) |
| Delta Temperature | 7-8° F. |
| Cycles of concentration | 3 |
| Tower water quality | Calcium hardness = 270-300 ppm as CaCO₃ |
| | Total alkalinity = 275-300 ppm as CaCO₃ |
| | TDS = 300-400 ppm |
| | pH = 8.3-8.8 |
| Makeup water | Potable city water |
| | Calcium hardness = 90-130 ppm as CaCO₃ |
| | TDS = 300-400 ppm |
| | pH = 7.2-7.4 |
| System metallurgy | Copper, brass, mild steel |
| | Cooling tower is galvanized steel |

The cooling system had been on a biocide program of 1,3-bromochloro-5,5-dimethylhydantoin (“BCDMH”) based briquettes. Use of BCDMH required long feed times of 5 to 8 hours to approach the target residual biocide concentration of 0.5 ppm free Cl₂, even though a booster pump had been installed to increase water flow through the bromine feeder to 18 gpm. The long feed times together with the fouling conditions presented in the tower led to high biocide consumption. The BCDMH was replaced with 1,3-dibromo-5,5-dimethylhydantoin (“DBDMH”) in the form of granules that have better dissolution rates and thus enabled higher target residual biocide concentration in a shorter period of time. Indeed, the use of DBDMH in this system led to over 1 ppm free Cl₂ after 30 minutes of feed. This residual was not persistent but declined within an hour. This decline was accompanied with the development of thick foam in the cooling tower basin indicating attack of biomass in the system. Subsequent doses on successive days gave essentially the same results. Over the next two months this dosage schedule was repeated every working day with a 30 minute feed time sufficient to provide a free Cl₂ residual of about 1 ppm. Undoubtedly the high surface area of the granular DBDMH together with it high bromine content allowed the plant to achieve the desired target residual in a short period of time. At the end of a two-month trial the condenser/chiller system was reported to be operating at design capacity and a shut down for manual cleaning was deemed unnecessary. The switch to DBDMH not only led to improved system performance but also resulted in a reduction in solid biocide consumption of about 70%. The granules of DBDMH employed in the trial quickly provided a desired free residual as Cl₂ in contrast to the BCDMH which required much longer feed times to achieve a much lower free residual as Cl₂. Table 2 summarizes the results of this trial.

TABLE 2
Summary of the Manufacturing Plant Field Trial

| | Previous Program | | New Program | |
|---|---|---|---|---|
| Scale & corrosion | Molybdate/phosphonate/ | | Molybdate/phosphonate/ | |
| program | TT | | TT | |
| Biocide Program | BCDMH¹-based | | DBDMH² granules | |
| | briquettes | | | |
| Slug dose frequency | 6-7 | times per | 5 | times per |
| | | week | | week |
| Feed time | 5-8 | hours | 0.5 | hour |
| Free residual, as Cl₂ | <0.3 | ppm | >1.0 | ppm |
| Total residual, | 2-4 | ppm | 1-3 | ppm |
| as Cl₂ | | | | |
| ORP³/mV | 400-500 | | 500-600 | |
| Biocide product | 75-87 | pounds/week | 28 | pounds/week |
| consumption | | | | |

¹1,3-bromochloro-5,5-dimethylhydantoin
²1,3-dibromo-5,5-dimethylhydantoin
³Oxidation-Reduction Potential

### EXAMPLE 2

A study was conducted to compare the solubility and pH impact of DBDMH to other common biocides. Chlorine, bromine and pH measurements were conducted on solid biocides in contact with water in 1-quart narrow-mouth high density polyethylene (“HDPE”) bottles. The bottles were half-filled with the solid biocide and then either distilled (“DI”) water or cooling tower water (obtained from the cooling tower at the Albemarle Technical Center, Baton Rouge, La., prior to the daily slug dose of biocide) was introduced to completely wet the solid. The bottles were allowed to equilibrate overnight. The pH of the liquid phase samples was determined using a hand-held pH meter (Hach Model EC10) calibrated with pH 7 and pH 10 buffer solutions. Test results are seen in Table 3 where the solubility and pH of trichloroisocyanuric acid (“Triclor”), BCDMH, and 1,3-bromochloro-5,5-methylethylhydantoin (“BCMEH”) are compared to that of DBDMH. The study was conducted using granules having an average size in the range of about 6 U.S. Standard mesh to about 0.312 inch where at least about 90 wt % of the granules fell within this range.

TABLE 3
Selected Properties of Solid Oxidizing Biocides

| | | Solubility | |
|---|---|---|---|
| | | in Water, | pH (saturated |
| Biocide | Appearance | % (25° C.) | solution) |
| Triclor | White powder or tablets | 1.2% | 2.8 |
| BCDMH | White to off-white granules or | 0.2% | 3.5 |
| | tablets | | |
| BCMEH | White to off-white granules or | 0.2% | 3.5 |
| | tablets | | |
| DBDMH | White to off-white granules | 0.1% | 6.6 |

As may be determined from Table 3, DBDMH is less soluble than other common solid biocides. This property of DBDMH serves the worthwhile purpose of reducing the erosion rate when DBDMH is used in a feeder type having design characteristics herein described. Of particular interest is the observation that DBDMH does not lower the pH of the system in contrast to the other biocides tested.

### EXAMPLE 3

As noted above, an advantage of an embodiment of this invention provides for extended periods of time before replenishment of the solid biocides is required. This overcomes the handicap of industrial water systems using erosion-type flow-through feeders which must be frequently opened and refilled with biocide material. The aqueous solutions contained in the feeder can become saturated with the biocide. Since chlorine and bromine vapors can be generated from biocides in a low pH condition, tests were conducted on the bottles of biocide solution in Example 2 to measure chlorine and bromine levels in the vapor space above the solutions saturated with solid biocides. Chlorine and bromine measurements were conducted by sampling the vapor space for five minutes (NIOSH Sampling Method 6011) using a calibrated MSA high flow sampling pump set at 2 L/min. The samples were collected on a filter (SKC 225-9006) containing a 0.5 μm Teflon® prefilter and a specially cleaned 0.45 μm silver membrane filter and then quantitatively reduced using sodium thiosulfate. The anions (chloride or bromide) were determined by ion chromatography. The pH of the saturated solutions was also determined. These studies were conducted in both distilled (DI) water and a cooling tower (CT) water sample. The results of these measurements are shown in Table 4.

TABLE 4
Chlorine and Bromine Levels and pH Measurements
of Saturated Biocide Solutions

| | Chlorine and Bromine Levels | | | |
|---|---|---|---|---|
| | in ppm (Br₂/Cl₂) | | pH Measurements | |
| Biocide | DI Water | CT Water | DI Water | CT Water |
| DBDMH | <1/<1 | <1/<1 | 6.6 | 7.1 |
| BCDMH | 16/5 | 2/4 | 3.1 | 5.3 |
| Trichlor | <1/61 | <1/73 | 2.4 | 3.0 |

These results indicate that DBDMH generates low levels of bromine vapors in comparison to other solid biocides. Use of DBDMH should result in significantly less exposure of personnel to irritating bromine vapors and eliminate exposure to chlorine vapors when this invention is in use.

Whether the aqueous media to be treated with microbiocide is from a highly purified water source, from industrial water systems, or from recreational systems, it is understood that “aqueous” permits such commonly occurring impurities such as salts, organic impurities and the like.

Compounds referred to by chemical name or formula anywhere in this document, whether referred to in the singular or plural, are identified as they exist prior to coming into contact with another substance referred to by chemical name or chemical type (e.g., another component, a solvent, or etc.). It matters not what chemical changes, if any, take place in the resulting mixture or solution, as such changes are the natural result of bringing the specified substances together under the conditions called for pursuant to this disclosure. As an example, the phase “solution of at least one 1,3-dibromo-5,5-dialkylhydantoin” and phrases of similar import signify that just before being brought into contact with an aqueous medium such as water, at least one 1,3-dibromo-5,5-dialkylhydantoin referred to was the specified 1,3-dibromo-5,5-dialkylhydantoin. The phrase thus is a simple, clear way of referring to the solution, and it is not intended to suggest or imply that the chemical exists unchanged in the water. The transformations that take place are the natural result of bringing these substances together, and thus need no further elaboration.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

All documents referred to herein are incorporated herein by reference in toto as if fully set forth in this document.

This invention is susceptible to considerable variation in its practice. Therefore the foregoing description is not intended to limit, and should not be construed as limiting, the invention to the particular exemplifications presented hereinabove. Rather, what is intended to be covered is as set forth in the ensuing claims and the equivalents thereof as permitted as a matter of law.

That which is claimed is:

1. A method for forming an aqueous microbiocidal solution which method comprises passing water into a tank and upwardly through a bed of microbiocide disposed in the tank so that the aqueous microbiocidal solution is formed, the microbiocide being at least one 1,3-dibromo-5,5-dialkylhydantoinin which one of the alkyl groups is a methyl group and the other alkyl group contains in the range of 1 to about 4 carbon atoms, the microbiocide being in a non-powdery particulate form.

2. A method according to claim 1 wherein the 1,3-dibromo-5,5-dialkylhydantoin is 1,3-dibromo-5,5-dimethylhydantoin.

3. A method according to claim 1 wherein water is caused to flow through a portion of the bed.

4. A method according to claim 2 wherein the tank comprises a pressure sealable first port at an upper portion of the tank to allow replenishment of the microbiocide in the bed, the bed being disposed in a lower portion of the tank.

5. A method according to claim 4 wherein the passage of the water through the tank is from a dispensing second port in an upward direction of flow through the lower portion of the tank and through at least a portion of the bed, and thereafter in a substantially horizontal direction out of the tank through a third port disposed between the lower and the upper portions of the tank.

6. A method according to claim 5 wherein the 1,3-dibromo-5,5-dimethylhydantoin is in the form of granules.

7. A method according to claim 5 wherein the 1,3-dibromo-5,5-dimethylhydantoin is in the form of nuggets.

8. A method according to claim 5 wherein water is caused to flow intermittently through the tank in time cycles having periods of active flow alternated with static periods, such that the solution has a pH in the range of about 5 to about 8 when measured proximate in time to the beginning of the period of active flow.

9. A system for treating an aqueous medium with a microbiocide which system comprises:
(A) a vertically elongated tank comprising (i) an upper portion comprising a pressure sealable first port, (ii) a lower portion comprising a dispensing port, and (iii) a third port disposed between the upper and lower portions;
(B) a bed of microbiocide which microbiocide is comprised of a 1,3-dibromo-5,5-dialkylhydantoin in which one of the alkyl groups is a methyl group and the other alkyl group contains in the range of 1 to about 4 carbon atoms, which microbiocide is of a non-powdery particulate form; and
(C) water to be treated with the microbiocide;
wherein when the bed is disposed in the tank through the first port, water is dispensed into the tank through the second port to flow upwardly through at least a portion of the bed so as to form an aqueous solution comprising a microbiocidally effective amount of the microbiocide, which solution flows substantially horizontally out of the tank through the third port.

10. A system according to claim 9 wherein the first port is sized and configured to permit replenishment of the microbiocide in the bed.

11. A system according to claim 10 wherein the 1,3-dibromo-5,5-dialkylhydantoin is 1,3-dibromo-5,5-dimethylhydantoin.

12. A system according to claim 10 wherein the 1,3-dibromo-5,5-dimethylhydantoin is in the form of granules.

13. A system according to claim 10 wherein the 1,3-dibromo-5,5-dimethylhydantoin is in the form of nuggets.

14. A method for forming an aqueous microbiocidal solution which method comprises:
(A) disposing a bed of a microbiocide comprising a 1,3-dibromo-5,5-dialkylhydantoin in which one of the alkyl groups is a methyl group and the other alkyl group contains in the range of 1 to about 4 carbon atoms, which microbiocide is of a non-powdery particulate form, into a lower portion of a vertically elongated tank though a pressure sealable first port at an upper portion of the tank;
(B) dispensing water through a second port in the lower portion of the tank;
(C) passing the water upwardly through at least a portion of the bed so as to form an aqueous microbiocidal solution; and
(D) diverting the aqueous microbiocidal solution from its upward flow so that it flows substantially horizontally out of the tank through a third port of the tank;
wherein the solution which exits the third port of the tank comprises a microbiocidally effective amount of the microbiocide.

15. A method according to claim 14 comprising replenishing the bed through the first port.

16. A method according to claim 15 wherein the 1,3-dibromo-5,5-dialkylhydantoin is 1,3-dibromo-5,5-dimethylhydantoin.

17. A method according to claims 16 wherein the 1,3-dibromo-5,5-dimethylhydantoin is in the form of granules.

18. A method according to claim 16 wherein the 1,3-dibromo-5,5-dimethylhydantoin is in the form of nuggets.

19. In a method for sanitizing and disinfecting an aqueous medium by adding an effective sanitizing and disinfecting amount of a sanitizing and disinfecting agent to the aqueous medium, the improvement which comprises passing at least a portion of the water of the aqueous medium through or over a bed of the agent consisting essentially of granules of a 1,3-dibromo-5,5-dialkylhydantoin in which one of the alkyl groups is a methyl group and the other group contains in the range of 1 to about 4 carbon atoms, the granules having an average size in the range of from about 60 to about 6 U.S. Standard mesh, the bed being contained in a dispensing tank through which the portion of the water is passed.

20. A method according to claim 6 wherein the granules have typical properties of (a) an assay of 1,3-dibromo-5,5-dimethylhydantoin of at least about 98 wt %, (b) available bromine of about 11 wt %, (c) an average particle size range of about 6 U.S. Standard mesh to about 0.3 inch, (d) a solubility in water at about 68° F. of about 0.1 wt %, and (e) a pH of about 6.6 of a slurry in water of about 1 wt %.
